(19) United States
(12) Patent Application Publication
(10) Pub. No.: US 2026/0334960 A1
(43) Pub. Date: Oct. 29, 2026

(54) BUR GUIDE FOR JOINT FUSIONS

(71) Applicant: Acumed LLC, Hillsboro, OR (US)

(72) Inventor: Nathan Stone, Little Elm, TX (US)

(21) Appl. No.: 19/653,912

(22) Filed: Apr. 21, 2026

Related U.S. Application Data
(60) Provisional application No. 63/793,314, filed on Apr. 23, 2025.

(51) Int. Cl. A61B 17/17; A61B 17/00; A61B 17/32; A61B 90/00

(52) U.S. Cl. CPC A61B 17/1775; A61B 17/32; A61B 90/03; A61B 17/00234; A61B 2017/320052

(57) ABSTRACT

A bone cutting guide can include a base body couplable to a bone of a patient and a cutting slot including first and second side walls. The guide can include a first insert movable between a deployed position in which the first insert is positioned at least partially within the cutting slot to form a first gap defining a first osteotomy path and a retracted position in which the first insert is not positioned within the cutting slot. The guide can include a second insert movable between a deployed position in which the first insert is positioned at least partially within the cutting slot to form a second gap defining a second osteotomy path and a retracted position in which the first insert is not positioned within the cutting slot. The first gap and the second gap can be sized to receive a cutting tool such as a bur.

### INCORPORATION BY REFERENCE TO ANY PRIORITY APPLICATIONS

The application claims benefit under 35 U.S.C. § 119(e) to U.S. Provisional Patent Application Ser. No. 63/793,314, filed Apr. 23, 2025, entitled “BUR GUIDE FOR JOINT FUSIONS”, the disclosure of which is hereby incorporated by reference and made part of this specification as if set forth fully herein in its entirety. Any and all applications for which a foreign or domestic priority claim is identified in the Application Data Sheet as filed with the present application are hereby incorporated by reference under 37 CFR 1.57.

### BACKGROUND

Lapidus procedures refer to certain surgical procedures for the treatment of a bunion deformity. A bunion is a bony bump that is formed on the side of the big toe joint, which may cause a foot deformity. Lapidus procedures involve the correction of the foot structure by repositioning the poorly aligned first metatarsal bone to its proper position and fusing it to the medial cuneiform.

Lapidus procedures traditionally require full exposure of the first tarsometatarsal (“TMT”) joint. This full exposure means that traditionally an incision having a length of at least 3.5 cm is needed to complete the Lapidus procedure, which may leave a big scar, require more healing time, and have a risk of infection.

### SUMMARY

Traditional cutting guides used in Lapidus procedures require thin sawblades that remove a few millimeters of bone, which can be achieved by placing two slots close to one another. One such cutting guide system allows for translation of the cutting slot along an axis. Such a system allows for the adjustment of the amount of bone removed, but translation adjustments tend to be larger and more complex than simpler cutting guides. Furthermore, the process of ensuring that the cutting guides are set to cut an exact amount of bone requires extra time in the procedure, which leaves an opportunity for human error in setting the wrong amount of bone to resect, either taking too much or too little bone.

Another cutting guide system pins blocks to both sides of a joint with a cutting slot offset to one side. One cut is made, then one block is removed, rotated around 180 degrees, and refastened back onto the same positioning pins to offset the cut in the opposite direction for the second cut. Such a system requires additional instrumentation to ensure the wires are placed sufficiently parallel to allow the block to slide off and back on.

Another cutting guide system may be used for minimally invasive surgery (“MIS”) procedures (e.g., MIS joint fusion procedures or MIS osteotomy procedures), which often require multiple small incisions which necessitate the use of burs over sawblades. Burs for cutting through bone are typically around 2 mm in diameter, or larger. Thus, using the same approach to a cutting guide may remove more bone than desired. Cutting guides designed for use with burs typically have only one cutting slot and cut one side of the joint followed by compressing the joint and cutting the opposing side through the same cut slot. Such cutting guides have some variation in cut width or depth based on how much the joint is compressed and may be hindered if the first cut leaves any bit of a cortical wall lip that could prevent total compression of the joint. Such cutting guides also require compressing and cutting multiple times to ensure the second cut is cut to the correct depth.

The existing cutting guide systems described above do not allow for the retraction of a cutting guide from an incision. Existing systems including a viewing window may allow for the complete removal of a cutting guide to access the incision with the guide placed back over k-wires, if necessary. However, systems that include a viewing window are incompatible with MIS procedures. Accordingly, there is a need for a cutting guide system that allows for the retraction of a cutting guide from an incision, which may be more suitable for MIS procedures.

In light of the technical features set forth herein, and without limitation, in a first aspect of the disclosure in the present application, a bone cutting guide for cutting at least one of a first bone and a second bone of a patient includes a base body configured to be fixed to the first bone, a cutting slot configured to provide a guiding surface for cutting at least one of the first bone and the second bone, a first insert configured to retract out of the cutting slot, and a second insert configured to retract out of the cutting slot.

In another aspect of the disclosure in the present application, the cutting slot is configured to retract away from the incision.

In another aspect of the disclosure in the present application, the cutting slot is configured to mechanically lock.

In another aspect of the disclosure in the present application, the bone cutting guide further includes a tab configured to retract the cutting slot.

In another aspect of the disclosure in the present application, the cutting slot, the first insert, and the second insert are disposed on a pin affixed to the base body.

In another aspect of the disclosure in the present application, the base body includes a wall configured to obstruct the cutting slot, the first insert, and the second insert from full rotation about the base body.

In another aspect of the disclosure in the present application, the first insert includes a first lip configured to interact with the cutting slot, and the second insert includes a second lip configured to interact with the cutting slot.

In another aspect of the disclosure in the present application, the first insert includes a first fin configured to extend into the joint between the first bone and the second bone; and the second insert includes a second fin configured to extend into the joint between the first bone and the second bone.

In another aspect of the disclosure in the present application, the base body is configured to receive at least one k-wire, pin, tack, or other fixation device to fix the base body to the first bone.

In another aspect of the disclosure in the present application, the bone cutting guide further includes a second body configured to be fixed to the second bone.

In another aspect of the disclosure in the present application, the second body is translatable along a path and is configured to compress or distract a joint between the first bone and the second bone.

In another aspect of the disclosure in the present application, a first aperture is formed between the first insert and an opposing first side wall of the cutting slot when the first insert is positioned within the cutting slot and the second insert is retracted out of the cutting slot.

In another aspect of the disclosure in the present application, the first aperture defines a path for a first osteotomy.

In another aspect of the disclosure in the present application, a bone cutting device is positioned through the first aperture.

In another aspect of the disclosure in the present application, the bone cutting device is a bur.

In another aspect of the disclosure in the present application, a second aperture is formed between the second insert and an opposing second side wall of the cutting slot when the second insert is positioned within the cutting slot and the first insert is retracted out of the cutting slot.

In another aspect of the disclosure in the present application, the second aperture defines a path for a second osteotomy.

In another aspect of the disclosure in the present application, a bone cutting device is positioned through the second aperture.

In some aspects, the techniques described herein relate to a bone cutting guide including: a base body to be coupled to a first bone of a patient; a cutting slot including a first side wall and a second side wall; a first insert to be selectively transitioned between a deployed position in which the first insert is positioned at least partially within the cutting slot and a retracted position in which the first insert is not positioned within the cutting slot; and a second insert to be selectively transitioned between a deployed position in which the first insert is positioned at least partially within the cutting slot and a retracted position in which the first insert is not positioned within the cutting slot.

In some aspects, the techniques described herein relate to a bone cutting guide, wherein: when the first insert is in the deployed position and the second insert is in the retracted position, a first gap is formed between the first insert and the second sidewall, the first gap sized to receive a cutting tool and defining a first osteotomy path; and when the second insert is in the deployed position and the first insert is in the retracted position, a second gap is formed between the second insert and the first sidewall, the second gap sized to receive the cutting tool and defining a second osteotomy path.

In some aspects, the techniques described herein relate to a bone cutting guide, wherein the cutting tool is a bur.

In some aspects, the techniques described herein relate to a bone cutting guide, wherein the cutting slot can be be selectively transitioned between a deployed position in which the cutting slot is positioned proximate an incision site of the patient and a retracted position in which the cutting slot is positioned away from the incision site of the patient.

In some aspects, the techniques described herein relate to a bone cutting guide, including a locking mechanism to selectively retain the cutting slot in the deployed position, the locking mechanism extending from the base body to contact the cutting slot.

In some aspects, the techniques described herein relate to a bone cutting guide, wherein the cutting slot, the first insert, and the second insert are selectively repositionable relative to the base body while the base body remains coupled to the bone.

In some aspects, the techniques described herein relate to a bone cutting guide, wherein the cutting slot, the first insert, and the second insert are pivotably coupled to the base body.

In some aspects, the techniques described herein relate to a bone cutting guide, wherein the cutting slot, the first insert, and the second insert can pivot away from an incision site of the patient to provide access to the incision site.

In some aspects, the techniques described herein relate to a bone cutting guide, wherein the cutting slot, the first insert, and the second insert are pivotably coupled to the base body proximate a first end of each.

In some aspects, the techniques described herein relate to a bone cutting guide, further including a wall to limit a maximum degree of rotation of the cutting slot, the first insert, and the second insert relative to the base body.

In some aspects, the techniques described herein relate to a bone cutting guide, wherein: the first insert includes a first fin to contact the patient when the first insert is in the deployed position; and the second insert includes a second fin to contact the patient when the second insert is in the deployed position.

In some aspects, the techniques described herein relate to a bone cutting guide, wherein the first fin extends from the first insert proximate a free end of the first insert, the first fin extending in a rotational direction of the cutting slot relative to the first insert, and the second fin extends from the second insert proximate a free end of the second insert, the second fin extending in a rotational direction of the cutting slot relative to the second insert.

In some aspects, the techniques described herein relate to a bone cutting guide, wherein the first fin extends beyond a bottom surface of the cutting guide when the first insert is in the deployed position, and the second fin extends beyond the bottom surface of the cutting guide when the second insert is in the deployed position.

In some aspects, the techniques described herein relate to a bone cutting guide, wherein: the first insert includes a first retention feature to interact with the cutting slot to inhibit the first insert from transitioning toward the retracted position when the cutting tool is received in the first gap; and the second insert includes a second retention feature to interact with the cutting slot to inhibit the second insert from transitioning toward the retracted position when the cutting tool is received in the second gap.

In some aspects, the techniques described herein relate to a bone cutting guide, wherein the first osteotomy path and the second osteotomy path are parallel to each other.

In some aspects, the techniques described herein relate to a bone cutting guide, wherein the first osteotomy path defines a first cutting plane extending through the first bone of the patient, and wherein the second osteotomy path defines a second cutting plane extending through a second bone of the patient, the second bone of the patient forming a joint with the first bone.

In some aspects, the techniques described herein relate to a bone cutting guide, further including a second body to be coupled to a second bone of the patient, the second bone forming a joint with the first bone.

In some aspects, the techniques described herein relate to a bone cutting guide, wherein the second body is translatable along a shaft relative to the base body, the second body to translate toward the base body to distract the joint between the first bone and the second bone and to translate away from the base body to compress the joint between the first bone and the second bone.

In some aspects, the techniques described herein relate to a bone cutting guide, wherein: the first insert includes a first tab extending from a free end of the first insert; the second insert includes a second tab extending from a free end of the second insert; and the first tab and second tab extend beyond an outer periphery of the cutting slot.

In some aspects, the techniques described herein relate to a bone cutting guide, wherein the first insert extends in a first lateral direction and the second insert extends in a second lateral direction opposite the first lateral direction.

Any embodiment disclosed herein may be combined with any other embodiment unless specified otherwise.

Additional features and advantages of the disclosed method and apparatus are described in, and will be apparent from, the following Detailed Description and the Figures. The features and advantages described herein are not all-inclusive and, in particular, many additional features and advantages will be apparent to one of ordinary skill in the art in view of the figures and description. Moreover, it should be noted that the language used in the specification has been principally selected for readability and instructional purposes, and not to limit the scope of the inventive subject matter.

### BRIEF DESCRIPTION OF THE FIGURES

FIG. 1 is a top perspective view of a bone cutting guide system with a first insert and a second insert retracted out of a cutting slot, according to one aspect of the present disclosure.

FIG. 2 is a top view of a bone cutting guide system with a first insert and a second insert retracted out of a cutting slot, according to one aspect of the present disclosure.

FIG. 3 is a top view of a bone cutting guide system with a first insert positioned within a cutting slot and a second insert retracted out of the cutting slot, according to one aspect of the present disclosure.

FIG. 4 is a top perspective view of a bone cutting guide system with a first insert retracted out of a cutting slot and a second insert positioned within the cutting slot, according to one aspect of the present disclosure.

FIG. 5 is a bottom perspective view of a bone cutting guide system with a first insert, a second insert, and a cutting slot in a retracted position, according to one aspect of the present disclosure.

FIG. 6A is a side view of a bone cutting guide system affixed to at least one bone with a first insert retracted out of a cutting slot and a second insert positioned within the cutting slot, prior to an osteotomy, according to one aspect of the present disclosure.

FIG. 6B is a side view of a bone cutting guide system affixed to at least one bone with a first insert positioned within a cutting slot and a second insert retracted out of the cutting slot, after an osteotomy has been made, according to one aspect of the present disclosure.

FIG. 6C is a side view of a bone cutting guide system affixed to at least one bone with a cutting slot, a first insert, and a second insert pivoted away from incisions, according to one aspect of the present disclosure.

FIG. 6D is a perspective view of a bone cutting guide system affixed to at least one bone with a cutting slot, a first insert, and a second insert pivoted away from incisions, according to one aspect of the present disclosure.

FIG. 7 is a perspective view of a bone cutting guide system having a base body and a second body, according to one aspect of the present disclosure.

### DETAILED DESCRIPTION OF EXAMPLES

The present disclosure is directed to systems and methods for a (e.g., stationary) bone cutting guide. In some embodiments, the bone cutting guide (e.g., one or more components thereof such as a base and/or a cutting slot) can remain fixed relative to a target bone or joint, while one or more components of the bone cutting guide that define a cutting path for a bone cutting tool such as a bur (e.g., a cutting slot, a first insert, and/or a second insert) can be selectively repositioned. Maintaining the bone cutting guide in a stationary position relative to the target bone or joint may reduce the need for repeated repositioning of the cutting guide after initial fixation to the bone or joint.

As discussed above, a conventional Lapidus procedure may require an incision having a length of at least 3.5 cm, which may have various issues such as a big scar, more healing time, and infection risk. During such procedures, it is preferable to minimize the size and number of cuts necessary to perform the surgery while still providing accurate and effective treatment. In some embodiments, the disclosed bone cutting guide systems and methods may facilitate such procedures through a reduced-size incision without compromising the accuracy of treatment. In some embodiments, the bone cutting guide systems and methods disclosed herein are suitable for MIS procedures.

Aspects of the present disclosure may address the above-discussed issues in surgical procedures such as the conventional Lapidus procedure and MIS procedures, and/or have other advantages. For example, aspects of the present disclosure may provide a system (e.g., a stationary system) capable of enabling precise cuts, such as parallel cuts that may overlap, without the need for additional instrumentation to translate a cutting guide along a path. These precise cuts may provide the removal of less bone from the joint when completing joint preparation with a bur than in typical bone cutting guide systems that use burs and may additionally or alternatively allow for smaller incisions when completing joint preparation with a bur than in typical bone cutting systems that use saw blades. Additionally, the present disclosure allows for retraction of the cutting guide from an incision to allow for optimized access to the patient, improved visualization of the joint, and access to clear the joint of debris (e.g., increased access to the joint to clear debris). In some embodiments, the cutting guide can be retracted from the incision without detaching the cutting guide from the bone such that the orientation of the cutting guide relative to the bone can be maintained for any subsequent cuts after inspection of the joint.

While the below disclosure provides for bone cutting guide systems and methods for use in Lapidus procedures and MIS procedures, it will be appreciated that the bone cutting guide may be used in other surgical applications requiring precise bone osteotomies or joint fusions. The present disclosure may be used for any joint fusion where a bur is to be used to prepare the joint, for example, in MIS procedures or reduced incision procedures. In some embodiments, dimensions, shapes, and/or arrangements of the cutting guide components may be selected based on the anatomy of the target bone or joint of a particular procedure.

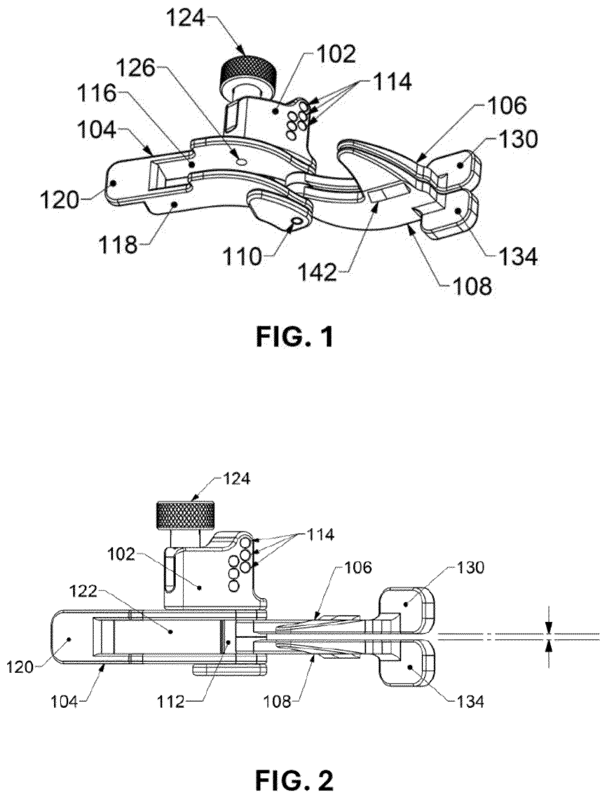

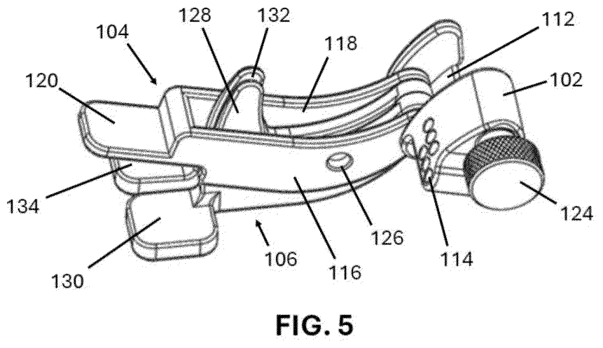

FIGS. 1 to 6D depict an exemplary bone cutting guide system 100. As shown in FIGS. 1 to 6D, the system 100 includes a base body 102, a cutting slot 104, a first insert 106 (e.g., first lateral insert, right insert), and a second insert 108 (e.g., second lateral insert, left insert). The base body 102 can function as an anchor structure that remains fixed relative to target bone or joint while the cutting slot 104 and/or one or both inserts 106, 108 are selectively repositioned. The first insert 106 and the second insert 108 are capable of being inserted between the walls of the cutting slot 104. The cutting slot 104 can be sized to receive both inserts 106, 108 simultaneously (for example, as shown in FIG. 5). In some embodiments, only one of the first insert 106 and the second insert 108 may be positioned within the cutting slot 104 during a given cutting step. When either one of the first insert 106 and the second insert 108 is positioned in the cutting slot 104, the resulting aperture between the insert (e.g., the respective insert 106, 108) and the opposing side of the slot (e.g., the cutting slot 104) allows a bur (or another cutting tool) to pass through to cut into a patient's bone. When one insert 106, 108 is disposed within the cutting slot 104 and the other insert 106, 108 is removed from the cutting slot 104, an aperture is formed within the cutting slot 104 which allows for passage of the bur. The formed aperture can be any of the apertures disclosed herein. In some embodiments, the formed aperture may limit or prevent lateral movement of the bur to assist in a substantially planar cut.

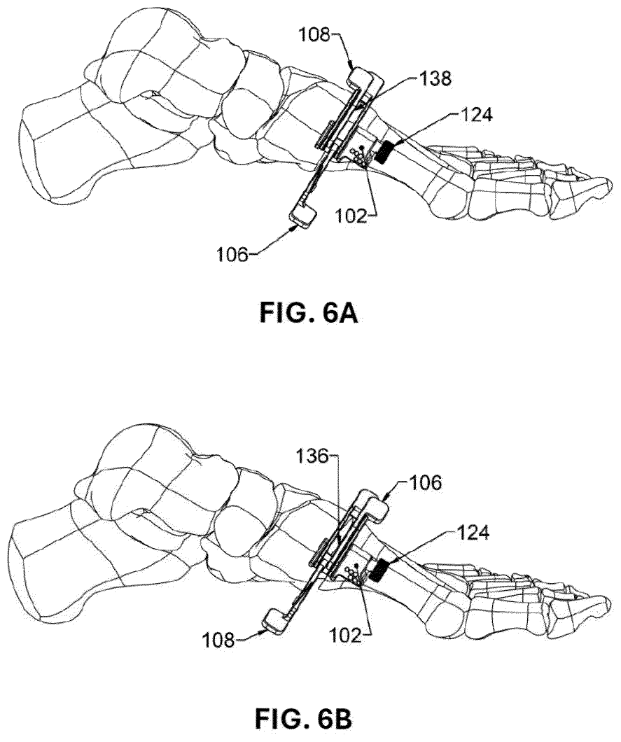

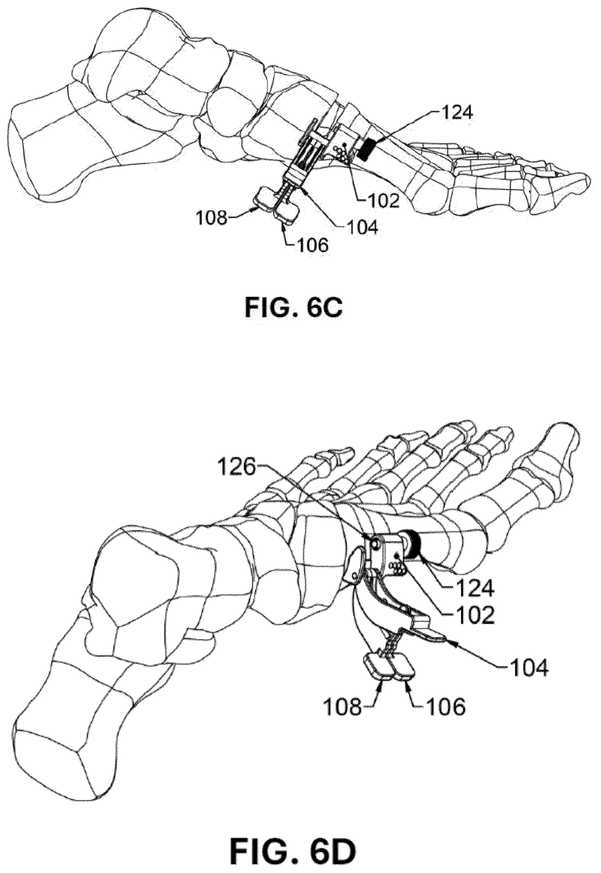

As shown in FIG. 1, the base body 102 of the bone cutting guide system 100 can be a loosely c-shaped body which facilitates the pivotable attachment of the cutting slot 104, the first insert 106, and the second insert 108. In some embodiments, the bone cutting guide system 100 can be partially or substantially L-shaped, J-shaped, arcuate, or linear. For example, the bone cutting guide system 100 (e.g., the cutting slot 104) can be shaped to partially or substantially conform to the shape of the target bone or joint on which the surgical procedure is being performed (e.g., the first metatarsal bone and/or the medial cuneiform of the first tarsometatarsal (“TMT”) joint as shown in FIGS. 6A-6D). In some embodiments, one or more portions (e.g., bottom surfaces) of the bone cutting guide system 100 (e.g., the cutting slot 104) can contact (e.g., rest on) the target bone or joint and/or tissue surrounding the target bone or joint during the surgical procedure which may help stabilize the bone cutting guide system 100.

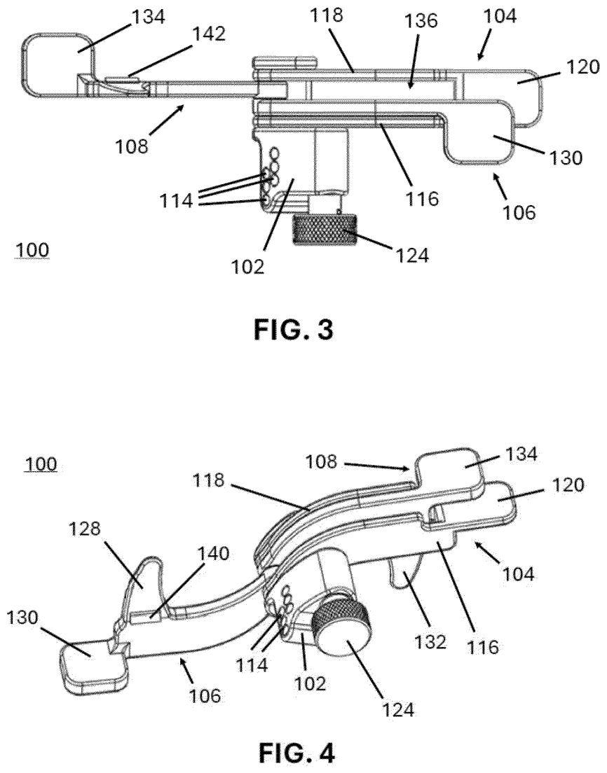

In some embodiments, the cutting slot 104, the first insert 106, and/or the second insert 108 can be rotatably (e.g., pivotably) coupled to the base body 102. The cutting slot 104, the first insert 106, and/or the second insert 108 can be rotatable (e.g., pivotable) between a deployed (e.g., cutting) position and retracted (e.g., withdrawn) position, for example, without moving the base body 102. In some embodiments, the cutting slot 104, the first insert 106, and/or the second insert 108 can pivotably couple to the base body 102 proximate an end (e.g., a proximate end) thereof. This may allow for greater access to an incision site and/or the targeted bone(s) when the cutting slot 104, the first insert 106, and/or the second insert 108 are pivoted to the retracted position. FIGS. 5, 6C, and 6D illustrate examples of the cutting slot 104, the first insert 106, and the second insert in the stowed or retracted position. FIGS. 3 and 6B illustrate examples of the first insert 106 and the cutting slot 104 in the deployed or extended position. FIGS. 4 and 6A illustrate examples of the second insert 108 and the cutting slot in the deployed or extended position.

In some embodiments, the cutting slot 104, the first insert 106, and/or the second insert 108 contact the target bone or joint and/or tissue surrounding the target bone or joint when in the deployed position, which may help stabilize the bone cutting guide system 100 during an osteotomy. In some embodiments, fewer component(s)or none of the cutting slot 104, the first insert 106, and/or the second insert 108 contacts the target bone or joint and/or tissue surrounding the target bone or joint when in the retracted position, which may allow for easier access to the incision site, for example, to clear the joint of debris after an osteotomy or to inspect the osteotomy.

In some embodiments, extending from one side of the base body 102 to the other is a strategically positioned pin 110 (see, e.g., FIG. 1). The pin 110 serves as a pivotal axis, enabling components affixed to the base body 102 (such as the cutting slot 104, the first insert 106, and/or the second insert 108) to rotate or pivot about the base body 102, for example, up to a wall 112 (see, e.g., FIG. 2) on the base body 102 which can be positioned parallel to the pin 110 and which can block full rotation of the cutting slot 104, the first insert 106, and/or the second insert 108 about the base body 102. In some embodiments, the wall 112 may define an abutment surface or stop that promotes repeatable positioning of one or more pivoting components (such as the cutting slot 104, the first insert 106, and/or the second insert 108) between the deployed position and the retracted position. For example, the wall 112 can limit how far (e.g., the degree/extent to which) the cutting slot 104, the first insert 106, and/or the second insert 108 can pivot in a first rotational direction (e.g., toward the target bone or joint, counterclockwise in the view of FIG. 1). In some embodiments, the wall 112 can additionally or alternatively limit how far (e.g., the degree/extent to which) the cutting slot 104, the first insert 106, and/or the second insert 108 can pivot in a second, opposite direction (e.g., away from the target bone or joint, clockwise in the view of FIG. 1).

The base body 102 can further include a fixation mechanism, allowing the base body 102 to be fixed (e.g., rotationally and/or translationally) to a patient's bone. In the illustrated embodiments, the fixation mechanism is a plurality of apertures 114, which are configured to receive a bone fastener. The bone fastener may be a pin, a screw, a tack, or a k-wire, and/or the like. In some embodiments, the base body 102 may include the plurality of apertures to fix the base body 102 on both sides of a patient's joint or in more than one bone of a patient. In some embodiments, the base body 102 may include one or more additional or alternative fixation mechanisms that are suitable for (e.g., removably) fixing the base body 102 to one or more bones of the patient such as clamps, straps, bands, hooks, staples, adhesives, or other components configured to fix the base body 102 relative to bone.

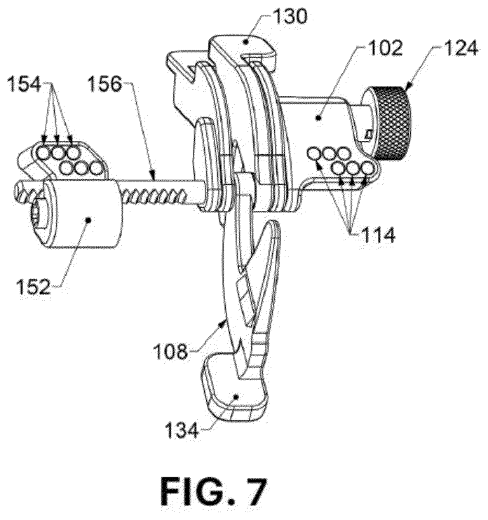

In other embodiments such as those illustrated by FIG. 7, a second body 152 is fixed to the opposing side of the patient's joint (e.g., a side of the patient's joint opposite the first body 102). The second body 152 can include one or more fixation mechanisms which may be similar or identical to the fixation mechanism(s) of the base body 102. The second body 152 may assist in stabilizing the bone cutting guide system 100 relative to the target bone or joint. In the illustrated embodiments, the fixation mechanism is a plurality of apertures 154, which are configured to receive a bone fastener. The bone fastener may be a pin, a screw, a tack, or a k-wire, and/or the like. The second body 152 is configured to translate along a path relative to the base body 102 to distract and compress the joint. For example, the second body 152 can be translated partially or substantially toward the base body 102 to compress the joint and/or partially or substantially away from the base body 102 to distract the joint. At least one of the base body 102 and the second body 152 may include threads as the mechanism to allow for distraction and compression of the joint (e.g., via translation of the second body 152 relative to the base body 102). In the illustrated embodiments, the path of translation is a shaft 156 extending from the pin 110. In some embodiments, translation of the second body 152 relative to the base body 102 may be controlled incrementally (e.g., via interaction with the threads and/or one or more protrusions or indentations in the shaft 156) to provide for more consistent and/or controlled compression and distraction of the joint.

In other embodiments, the bone fastener may be any other suitable fastening device that can (e.g., temporarily or removably) fix the base body 102 to the bone of a patient. In some examples, the bone fastener may include a hat that may prevent the bone fastener from penetrating into the bone deeper than a predetermined length (e.g., so that it does not penetrate into another bone unintentionally). In yet other embodiments, the fixation mechanism may be a clamp or any other suitable device that can (temporarily or removably) fix the base body 102 to the bone of a patient (e.g., medial cuneiform bone). It should be appreciated that the above-identified fixation mechanisms are exemplary and other fixation mechanisms may be added or omitted in other embodiments of the bone cutting guide system 100.

The base body 102 can further include a locking mechanism as described in further detail below. The locking mechanism may selectively maintain the cutting slot 104 in a desired cutting position relative to the base body 102. For example, the locking mechanism may selectively prevent or inhibit the cutting slot 104 from pivoting relative to the base body 102 during a cutting procedure.

The cutting slot 104 provides a guide for cutting at least one of the first bone and the second bone of the patient. In some embodiments, the first bone can be a first bone of a joint on which a cutting procedure is being performed, for example, the first metatarsal or the medial cuneiform. The second bone can be a second bone of the joint opposite the first bone, for example, the other of the first metatarsal or the medial cuneiform. The cutting slot 104 includes a first side wall 116, a second side wall 118, and/or a cutting slot movement tab 120 extending from the first side wall 116 and the second side wall 118, for example, on an end furthest from the pin 110. The cutting slot movement tab 120 can extend away from the cutting slot 104 (e.g., a distal end of the cutting slot 104). As shown in illustrated embodiments, the first side wall 116 and the second side wall 118 may vary in shape. The area contained within the first side wall 116, the second side wall 118, and the cutting slot movement tab 120 define an aperture 122 as shown in FIG. 2. The aperture 122 provides a guide for a bone cutting device. Examples of the bone cutting device may include a bur, a blade, an osteotome, and a reciprocating saw. While the bone cutting guide system 100 is described primarily herein as being used with a bur, it will be appreciated from the disclosure herein that other bone cutting devices such as a blade, an osteotome, and/or a reciprocating saw can be used with the bone cutting guide system 100.

The cutting slot 104 (e.g., the first side wall 116, the second side wall 118, and/or the cutting slot movement tab 120) can be pivoted relative to the base body 102. For example, the cutting slot 104 can be pivotable about the pin 110. For example, a user can interact with (e.g., grasp, pull, push) the cutting slot movement tab 120 to pivot the cutting slot 104 between the deployed and stowed positions. In embodiments that include a wall 112 on the base body 102 such as the illustrated embodiments, the cutting slot 104 is pivotable up to the wall 112 which blocks full rotation of the cutting slot 104. In this regard, after osteotomies are made, the cutting slot 104 can pivot away from the osteotomies allowing for optimized access to the patient, improved visualization of the joint, and access to clearing the joint of debris, which can be especially useful in MIS procedures. Advantageously, because the cutting slot 104 is pivotable about the pin 110, the fixation mechanism of the base body 102 does not need to be moved to provide a surgeon access to the surgical area when pivoting the cutting slot 104 away from the incision. Thus, positioning the fixation mechanism of the base body 102 near the pin 110 may provide the surgeon with as much access to the incision as possible when the cutting slot 104 is pivoted away from the incision.

As shown in the illustrated embodiments, a surgeon may move the cutting slot 104 by utilizing the cutting slot movement tab 120. The use of the cutting slot movement tab 120 enables the cutting slot 104 to be easily moved without disrupting the procedure. In other embodiments, the cutting slot 104 does not include a cutting slot movement tab 120.

The base body 102 can further include a locking mechanism 124 to block the cutting slot 104 from moving when in use (e.g., during a cutting procedure). For example, the locking mechanism 124 can maintain the cutting slot 104 in the deployed position. The locking mechanism 124 may be coupled to (e.g., extend from) the base body 102. The locking mechanism 124 can selectively couple (e.g., fix) the cutting slot 104 to the base body 102 to selectively prevent or inhibit movement of the cutting slot 104. Preventing or inhibiting movement of the cutting slot 104 relative to the base body 102 may additionally prevent or inhibit movement of the cutting slot 104 relative to the target bone or joint. The locking mechanism 124 may be a pin, a detent, a catch or clasp, a cam lock, a screw, a tack, or any other mechanism capable of inhibiting, preventing, or obstructing the cutting slot 104 from further movement (e.g., pivoting), thereby enhancing the operational stability and safety of the bone cutting guide system 100. It should be appreciated that the above-identified locking mechanisms 124 are exemplary and other locking mechanisms 124 may be added or omitted in other embodiments of the bone cutting guide system 100.

In the illustrated embodiments, the locking mechanism 124 is a plunger threaded through the base body 102. When the plunger of the locking mechanism 124 is disposed within a locking mechanism aperture 126 of the cutting slot 104, the plunger blocks or inhibits the cutting slot 104 from rotating about the pin 110, for example, toward the retracted position. The locking mechanism aperture 126 can be formed in (e.g., extend through) the first or second sidewall 116, 118 of the cutting slot 104.

The bone cutting guide system 100 includes a first insert 106 and a second insert 108. In some embodiments, the first insert 106 and the second insert 108 can enable precise overlapping incision cut guides for joint preparation in Lapidus procedures and MIS procedures. The first insert 106 can include a body largely mimicking the shape of the cutting slot 104 with a first fin 128 extending outwards from the body of the first insert 106 and a first insert movement tab 130 for rotating the first insert 106 about the pin 110. The first fin 128 can extend from a bottom of the first insert 106. In some embodiments, the first fin 128 can extend from or proximate an end (e.g., a free end) of the first insert 106. The first fin 128 can extend partially or substantially in the first rotational direction (e.g., toward the target bone or joint, toward the cutting slot 104, counterclockwise in the view of FIG. 1). The first insert movement tab 130 can extend from or proximate an end (e.g., the free end) of the first insert 106. In some embodiments, the first insert movement tab 130 can extend laterally and/or longitudinally away from the first insert 106 (e.g., a body thereof). The second insert 108 can symmetrically mirror the first insert 106. For example, the second insert 108 can mirror the first insert 106 across a longitudinal axis of the cutting slot 104. The second insert 108 can include a body largely mimicking the shape of the cutting slot 104 with a second fin 132 extending outwards from the body of the second insert 108 (e.g., partially or substantially in a direction of the cutting slot 104) and a second insert movement tab 134 for rotating the second insert 108 about the pin 110. The second fin 132 can extend from a bottom of the second insert 108. In some embodiments, the second fin 132 can extend from or proximate an end (e.g., a free end) of the second insert 108. The second fin 132 can extend partially or substantially in the first rotational direction (e.g., toward the target bone or joint, toward the cutting slot 104, counterclockwise in the view of FIG. 1). The second insert movement tab 134 can extend from or proximate an end (e.g., the free end) of the second insert 108. In some embodiments, the second insert movement tab 134 can extend laterally and/or longitudinally away from the second insert 108 (e.g., a body thereof).

When in use during a surgical procedure, the first fin 128 may contact the patient and provide alignment of the first insert 106 with a patient's joint or bone. This may include extending into at least one of a patient's joints. Additionally, when in use during a surgical procedure, the second fin 132 may contact the patient and provide alignment of the second insert 108 with a patient's joint or bone. This may include extending into at least one of a patient's joints. The first fin 128 and/or the second fin 132 may extend beyond the cutting slot 104 (e.g., beyond a bottom surface of the cutting slot 104) when the first insert 106 and the second insert 108, respectively, are in the deployed position. In some embodiments, only one of the first fin 128 and the second fin 132 may contact the patient at a given time, for example, depending on which insert 106, 108 is disposed in the cutting slot 104 (e.g., in the deployed position). In other embodiments, the first insert 106 and the second insert 108 do not include the first fin 128 and the second fin 132, respectively.

The first insert 106 and the second insert 108 are retractable from the cutting slot 104 and are pivotable about the pin 110. In embodiments that include a wall 112 on the base body 102 such as the illustrated embodiments, the first insert 106 and the second insert 108 are pivotable up to the wall 112 which blocks full rotation of both the first insert 106 and the second insert 108 (e.g., away from the cutting slot 104). In this regard, at any point in the procedure, at least one of the first insert 106, the second insert 108, and the cutting slot 104 can pivot away from the incisions allowing for optimized access to the patient, improved visualization of the joint, and access to clearing the joint of debris, which may be especially useful in MIS procedures.

Advantageously, because the first insert 106, the second insert 108, and the cutting slot 104 are pivotable about the pin 110, the fixation mechanism of the base body 102 does not need to be moved to provide a surgeon access to the surgical area. Instead, the first insert 106 or the second insert 108 may be moved by the first insert movement tab 130 or the second insert movement tab 134, respectively, and the cutting slot 104 may be moved by the cutting slot movement tab 120. In the illustrated embodiments, each of the first insert movement tab 130 and the second insert movement tab 134 overhangs the cutting slot 104, allowing for quick and easy movement of the first insert 106 and the second insert 108. In other embodiments, the first insert 106 and the second insert 108 do not include a first insert movement tab 130 and a second insert movement tab 134.

When either the first insert 106 or second insert 108 is disposed within the cutting slot 104, the aperture formed by the gap between the respective insert 106, 108 and an opposing wall 116, 118 of the cutting slot 104 defines a path for a first osteotomy. For example, when the first insert 106 is positioned within the cutting slot 104 but the second insert 108 is not positioned within the cutting slot 104, an offset first insert aperture 136 is formed between the first insert 106 and the second side wall 118, as shown in FIGS. 3 and 6B. The first insert aperture 136 then defines a path for an osteotomy by a bone cutting device of a patient's joint or bone. In another example, when the second insert 108 is positioned within the cutting slot 104 but the first insert 106 is not positioned within the cutting slot 104, an offset second insert aperture 138 is formed between the second insert 108 and the first side wall 116, as shown in FIG. 6A. The second insert aperture 138 then defines a path for an osteotomy by a bone cutting device of a patient's joint or bone. In some embodiments, the first insert 106 and the second insert 108 are sized such that a first osteotomy made via a path defined by the first insert aperture 136 and a second osteotomy made via a path defined by the second insert aperture 138 are either spaced apart, adjacent or overlapping. In some embodiments, the bur can substantially or completely fill a width of the first insert aperture 136 and/or the second insert aperture 138. For example, the bur can have a diameter that is equal or substantially similar to a width of the first and/or second insert apertures 136, 138. This may encourage or force the bur to move in a substantially planar direction when moving along the first and/or second insert apertures 136, 138 to cut a target bone, thereby creating a planar cut.

In the illustrated embodiments, the first insert 106 includes a first lip 140 configured to interact with the first side wall 116 of the cutting slot 104 to minimize movement of the first insert 106 while a bone cutting device is in use. For example, the first lip 104 may prevent or inhibit the first insert 106 from pivoting away from the cutting slot 104 when in the deployed position (e.g., toward the retracted position). During a surgical procedure, a bone cutting device making an osteotomy in the first insert aperture 136 may contact the first insert 106, pushing the first insert 106 against the first side wall 116 of the cutting slot 104 (e.g., when the bur substantially or completely fills a width of the first insert aperture 136). If the first lip 140 is positioned below the first side wall 116, the first lip 140 obstructs the first insert 106 from movement (e.g., relative to the cutting slot 104) by hooking under the edge of the cutting slot 104. If the first lip 140 is positioned against the first side wall 116, the near perpendicular force of the first lip 140 against the first side wall 116 supports a frictional fit between the first insert 106 and the first side wall 116 which may prevent or inhibit movement of the first insert 106 relative to the cutting slot 104.

Likewise, in the illustrated embodiments, the second insert 108 includes a second lip 142 configured to interact with the second side wall 118 of the cutting slot 104 to minimize movement of the second insert 108 while a bone cutting device is in use. For example, the second lip 142 may prevent or inhibit the second insert 108 from pivoting away from the cutting slot 104 (e.g., toward the retracted position). During a surgical procedure, a bone cutting device making an osteotomy in the second insert aperture 138 may contact the second insert 108, pushing the second insert 108 against the second side wall 118 of the cutting slot 104 (e.g., when the bur substantially or completely fills a width of the second insert aperture 138). If the second lip 142 is positioned below the second side wall 118, the second lip 142 obstructs the second insert 108 from movement (e.g., relative to the cutting slot 104) by hooking under the edge of the cutting slot 104. If the second lip 142 is positioned against the second side wall 118, the near perpendicular force of the second lip 142 against the second side wall 118 supports a frictional fit between the second insert 108 and the second side wall 118 which may prevent or inhibit movement of the first insert 106 relative to the cutting slot 104.

It should be appreciated that the illustrated first lip 140 and second lip 142 are exemplary and other first lip 140 and second lip 142 configurations may be added or omitted in other embodiments of the bone cutting guide system 100. In some embodiments, the first insert 106 and/or the second insert 108 can include one or more retention features in addition to or alternatively to the first lip 140 and/or the second lip 142. Such retention features may include, for example, detents, snap-fits, interference features, magnetic elements, resilient engagements, latches, catches, frictional interfaces, spring-biased elements, or other structures configured to inhibit or prevent unintended movement of the first insert 106 and/or the second insert 108 relative to the cutting slot 104 during a cutting procedure. In other embodiments, the first insert 106 and second insert 108 do not include a first lip 140 and a second lip 142, respectively.

In other embodiments, the first insert 106 and the second insert 108 are pivotable about separate pins. By the addition of a secondary hinge mechanism, the first insert 106 and second insert 108 could define non-parallel cutting slots such as wedge cuts performed at a predetermined angle.

In yet other embodiments, the first insert 106 and the second insert 108 are not pivotable but instead slide against a component such as the base body 102 to retract away from the incision. Alternatively, the first insert 106 and the second insert 108 slide between the base body 102 fixed to a first bone and a second body (e.g., such as second body 152) configured to be fixed to a second bone.

FIGS. 6A to 6D illustrate an exemplary procedure utilizing the bone cutting guide system 100. In FIG. 6A, the bone cutting guide system 100 is aligned with the joint (e.g., the first tarsometatarsal joint as shown in FIGS. 6A-6D) and pinned to the bone (e.g., the first metatarsal as shown in FIGS. 6A-6D) percutaneously via a plurality of pins threaded through the base body 102. Alignment may be done using an additional device, or via the insertion of one of the first insert 106 or second insert 108 into a patient's joint. The cutting slot 104 and/or either the first insert 106 or the second insert 108 are positioned against a patient's bones and/or joint while the other of the first insert 106 and the second insert 108 is retracted away from the cutting slot 104 and patient. The insert 106, 108 selected for joint alignment determines which side of the joint will be cut first. In the embodiment illustrated by FIG. 6A, the cutting slot 104 and the second insert 108 are positioned against a patient's bones and joint (e.g., in the deployed position), while the first insert 106 is retracted away from the cutting slot 104 and patient (e.g., in the retracted position). The placement of the second insert 108 in the cutting slot 104 creates the second insert aperture 138 which can create a path for a first osteotomy.

With the bone cutting guide system 100 pinned in place to the patient and the cutting slot 104 piece locked down via the locking mechanism 124, fluoroscopy can be used to ensure alignment with the joint. The first cut may be made using a bone cutting device, such as a bur, through the cutting slot 104 while the second insert 108 is in place. For example, the first cut can be made through the second insert aperture 138 between the second insert 108 and the cutting slot 104 (e.g., the first sidewall 116 thereof).

Once a first osteotomy is made, the cutting slot 104 and the insert 106, 108 positioned against a patient's bones and/or joint are pivoted away from the patient so that a surgeon can review the osteotomy and clear the joint of any debris. Then, the cutting slot 104 and opposite insert 106, 108 are positioned against the patient. As shown in FIG. 6B, the cutting slot 104 and first insert 106 are positioned against a patient's bones and joint (e.g., in the deployed position), while the second insert 108 is retracted away from the cutting slot 104 and patient (e.g., in the retracted position). The placement of the first insert 106 creates the first insert aperture 136 which aligns with a path for a second osteotomy and is wide enough for a bone cutting device to fit. Because the first insert 106 and second insert 108 are pivotable about the same pin, the new osteotomy path can be parallel to the first osteotomy path on the opposing bone of the joint.

The first insert aperture 136 and the second insert aperture 138 are sized to allow the cutting device to pass through freely (e.g., partially or substantially freely, with minimal or no friction) while restricting movement to a planar cut. In some embodiments, the cutting slot 104, the first insert 106, and/or the second insert 108 can flex to accommodate the cutting device, for example, if the cutting device has a width (e.g., diameter) greater than a width of the first or second apertures 136, 138. If the first insert 106 and the second insert 108 are adjacent to one another, the first insert aperture 136 and the second insert aperture 138 will be adjacent. If a gap (e.g., lateral gap) exists between the first insert 106 and the second insert 108, the first insert aperture 136 and the second insert aperture 138, and the cutting planes those apertures 136, 138 define can overlap one another by the amount of the gap.

Returning to FIG. 6B, when the cutting slot 104 and first insert 106 are positioned against the patient and the second insert 108 is retracted away from the patient, a second osteotomy is made using a bone cutting device through the path created by the first insert aperture 136. Fore example, the second osteotomy can be made through the first insert aperture 136 between the first insert 106 and the cutting slot 104 (e.g., the second sidewall 118 thereof). As shown in FIGS. 6C and 6D, once the second osteotomy is made, the cutting slot 104 and the first insert 106 are pivoted away from the patient so that a surgeon can review the osteotomy and clear the joint of any debris. The joint may additionally be compressed with both the first insert 106, the second insert 108, and/or the cutting slot 104 retracted out of the patient's joint. As the bone cutting guide system 100 is attached to the patient only on one side of the joint, the joint can be distracted or compressed as necessary without removing the bone cutting guide system 100. After the joint is prepared for fusion, the bone cutting guide system 100 is then removed from the patient. Fixation of the first bone to the second bone may be completed with the cutting guide system 100 attached to the bone or after it has been removed.

It should be appreciated that the above-identified procedure shown in FIGS. 6A to 6D are exemplary and other procedures may be performed using the bone cutting guide system 100. For example, the order of creating the first and second cuts (e.g., osteotomies) using the first and second inserts 106, 108 described above can be reversed.

Although this disclosure has been described in the context of certain embodiments and examples, it will be understood by those skilled in the art that the disclosure extends beyond the specifically disclosed embodiments to other alternative embodiments and/or uses and obvious modifications and equivalents thereof. In addition, while several variations of the embodiments of the disclosure have been shown and described in detail, other modifications, which are within the scope of this disclosure, will be readily apparent to those of skill in the art. It is also contemplated that various combinations or sub-combinations of the specific features and aspects of the embodiments may be made and still fall within the scope of the disclosure. For example, features described above in connection with one embodiment can be used with a different embodiment described herein and the combination still fall within the scope of the disclosure. It should be understood that various features and aspects of the disclosed embodiments can be combined with, or substituted for, one another in order to form varying modes of the embodiments of the disclosure. Thus, it is intended that the scope of the disclosure herein should not be limited by the particular embodiments described above. Accordingly, unless otherwise stated, or unless clearly incompatible, each embodiment of this invention may comprise, additional to its essential features described herein, one or more features as described herein from each other embodiment of the invention disclosed herein.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, 0.1 degree, or otherwise.

In understanding the scope of the present invention, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Also, the terms “part,” “section,” “portion,” “member” or “element” when used in the singular can have the dual meaning of a single part or a plurality of parts.

When introducing elements of various embodiments, the articles “a,” “an,” “the,” and “said” are intended to mean that there are one or more of the elements. The terms “comprising,” “including,” and “having” are intended to be inclusive and mean that there may be additional elements other than the listed elements.

The examples and embodiments disclosed herein are to be construed as merely illustrative and not a limitation of the scope of the present disclosure in any way. It will be apparent to those having skill in the art that changes may be made to the details of the above-described embodiments without departing from the underlying principles discussed. In other words, various modifications and improvements of the embodiments specifically disclosed in the description above are within the scope of the appended claims. For example, any suitable combination of features of the various embodiments described is contemplated.

What is claimed is:

1. A bone cutting guide comprising:
a base body configured to be coupled to a first bone of a patient;
a cutting slot comprising a first sidewall and a second sidewall;
a first insert configured to be selectively transitioned between a deployed position in which the first insert is positioned at least partially within the cutting slot and a retracted position in which the first insert is not positioned within the cutting slot; and
a second insert configured to be selectively transitioned between a deployed position in which the first insert is positioned at least partially within the cutting slot and a retracted position in which the first insert is not positioned within the cutting slot.

2. The bone cutting guide of claim 1, wherein:
when the first insert is in the deployed position and the second insert is in the retracted position, a first gap is formed between the first insert and the second sidewall, the first gap sized to receive a cutting tool and defining a first osteotomy path; and
when the second insert is in the deployed position and the first insert is in the retracted position, a second gap is formed between the second insert and the first sidewall, the second gap sized to receive the cutting tool and defining a second osteotomy path.

3. The bone cutting guide of claim 2, wherein the cutting tool is a bur.

4. The bone cutting guide of claim 1, wherein the cutting slot is configured to be selectively transitioned between a deployed position in which the cutting slot is positioned proximate an incision site of the patient and a retracted position in which the cutting slot is positioned away from the incision site of the patient.

5. The bone cutting guide of claim 4, comprising a locking mechanism configured to selectively retain the cutting slot in the deployed position, the locking mechanism extending from the base body to contact the cutting slot.

6. The bone cutting guide of claim 1, wherein the cutting slot, the first insert, and the second insert are selectively repositionable relative to the base body while the base body remains coupled to the bone.

7. The bone cutting guide of claim 6, wherein the cutting slot, the first insert, and the second insert are pivotably coupled to the base body.

8. The bone cutting guide of claim 7, wherein the cutting slot, the first insert, and the second insert is configured to pivot away from an incision site of the patient to provide access to the incision site.

9. The bone cutting guide of claim 7, wherein the cutting slot, the first insert, and the second insert are pivotably coupled to the base body proximate a first end of each.

10. The bone cutting guide of claim 7, further comprising a wall configured to limit a maximum degree of rotation of the cutting slot, the first insert, and the second insert relative to the base body.

11. The bone cutting guide of claim 1, wherein:
the first insert comprises a first fin configured to contact the patient when the first insert is in the deployed position; and
the second insert comprises a second fin configured to contact the patient when the second insert is in the deployed position.

12. The bone cutting guide of claim 11, wherein the first fin extends from the first insert proximate a free end of the first insert, the first fin extending in a rotational direction of the cutting slot relative to the first insert, and the second fin extends from the second insert proximate a free end of the second insert, the second fin extending in a rotational direction of the cutting slot relative to the second insert.

13. The bone cutting guide of claim 11, wherein the first fin extends beyond a bottom surface of the bone cutting guide when the first insert is in the deployed position, and the second fin extends beyond the bottom surface of the bone cutting guide when the second insert is in the deployed position.

14. The bone cutting guide of claim 2, wherein:
the first insert comprises a first retention feature configured to interact with the cutting slot to inhibit the first insert from transitioning toward the retracted position when the cutting tool is received in the first gap; and
the second insert comprises a second retention feature configured to interact with the cutting slot to inhibit the second insert from transitioning toward the retracted position when the cutting tool is received in the second gap.

15. The bone cutting guide of claim 2, wherein the first osteotomy path and the second osteotomy path are parallel to each other.

16. The bone cutting guide of claim 2, wherein the first osteotomy path defines a first cutting plane extending through the first bone of the patient, and wherein the second osteotomy path defines a second cutting plane extending through a second bone of the patient, the second bone of the patient forming a joint with the first bone.

17. The bone cutting guide of claim 2, further comprising a second body configured to be coupled to a second bone of the patient, the second bone forming a joint with the first bone.

18. The bone cutting guide of claim 17, wherein the second body is translatable along a shaft relative to the base body, the second body configured to translate toward the base body to distract the joint between the first bone and the second bone and configured to translate away from the base body to compress the joint between the first bone and the second bone.

19. The bone cutting guide of claim 1, wherein:
the first insert comprises a first tab extending from a free end of the first insert;
the second insert comprises a second tab extending from a free end of the second insert; and
the first tab and second tab extend beyond an outer periphery of the cutting slot.

20. The bone cutting guide of claim 17, wherein the first insert extends in a first lateral direction and the second insert extends in a second lateral direction opposite the first lateral direction.
